SEQUENCE LISTING (17 sequences)

SEQ ID NO: 1 - protein, 106 aa, synthetic construct
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 11 aa, synthetic construct
GTELSVRAKPS

SEQ ID NO: 5 - protein, 118 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 6 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 347 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 5 aa, synthetic construct
GGGGS

SEQ ID NO: 10 - protein, 476 aa, synthetic construct
MEFGLSWLFLVAILKGVQCEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKDEL

SEQ ID NO: 11 - protein, 240 aa, synthetic construct
MKYLLPTAAAGLLLLAAQPAMADIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECKDEL

SEQ ID NO: 12 - protein, 10 aa, synthetic construct
GYTFTNYGMN

SEQ ID NO: 13 - protein, 17 aa, synthetic construct
WINTYTGEPTYAADFKR

SEQ ID NO: 14 - protein, 14 aa, synthetic construct
YPHYYGSSHWYFDV

SEQ ID NO: 15 - protein, 11 aa, synthetic construct
SASQDISNYLN

SEQ ID NO: 16 - protein, 7 aa, synthetic construct
FTSSLHS

SEQ ID NO: 17 - protein, 9 aa, synthetic construct
QQYSTVPWT

(19) United States
(12) Patent Application Publication
(10) Pub. No.: US 2025/0367255 A1
(43) Pub. Date: Dec. 4, 2025

(54) Enhancement Of CD47 Blockade Therapy With Anti-VEGF Agents

(71) Applicant: PFIZER INC., New York, NY (US)

(72) Inventors: Gloria Hoi Ying LIN, Oakville, Ontario (CA); Robert Adam UGER, Richmond Hill (CA); Mark Michael WONG, Toronto, Ontario (CA)

(73) Assignee: Pfizer Inc., New York, NY (US)

(21) Appl. No.: 18/708,093

(22) PCT Filed: Nov. 1, 2022
(86) PCT No.: PCT/IB2022/060516

§ 371 Date: May 7, 2024

Related U.S. Application Data
(60) Provisional application No. 63/277,101, filed on Nov. 8, 2021.

(51) Int. Cl. A61K 38/17; A61K 39/00; A61K 39/395; A61P 35/00

(52) U.S. Cl. CPC A61K 38/177; A61K 39/39558; A61P 35/00; A61K 2039/505

(57) ABSTRACT

Various forms of cancer and other diseases are treated using a medicinal combination of a SIRPαFc to block binding with CD47, and an anti-VEGF agent such as humanized antibody bevacizumab to control vascularization.

### FIELD

This invention relates to methods of using an agent that blocks the CD47/SIRPα interaction. More particularly, the invention relates to methods and means that, in combination, are useful for improving cancer therapy.

### BACKGROUND

Cancer cells can be targeted for destruction by antibodies that bind to cancer cell antigens, and through recruitment and activation of macrophages by way of Fc receptor binding to the Fc portion of that antibody. Binding between CD47 on cancer cells and SIRPα on macrophages transmits a “don't eat me” signal that enables many tumour cells to escape destruction by macrophages. It has been shown that inhibition of the CD47/SIRPα interaction (CD47 blockade) will allow macrophages to “see” and destroy the target CD47+ cancer cell. The use of SIRPα-based agents to treat cancer by CD47 blockade is described in international Patent Publication No. WO2010/130053.

In international Patent Publication No. WO2014/094122, protein drugs that inhibit the interaction between CD47 and SIRPα are provided. These CD47 blocking agents comprise a form of human SIRPα that binds CD47, and incorporate a particular region of its extracellular domain linked with a particularly useful form of an IgG1-based Fc region, or an IgG4-based Fc region. In this form, the SIRPαFc agent shows dramatic effects on the viability and vitality of cancer cells that present with a CD47+ phenotype. The effect is seen particularly in acute myelogenous leukemia (AML) cells, and in many other types of cancer including both liquid (blood) and solid forms. A form of SIRPα having significantly altered primary structure and enhanced CD47 binding affinity is described in international Patent Publication No. WO2013/109752. Still other useful forms of SIRPα include those that are bispecific and have anti-cancer proteins fused therewith.

Other CD47 blocking agents have been described in the literature and these include various CD47 antibodies (e.g., U.S. Pat. No. 8,562,997 (Stanford), and international Patent Publication No. WO2014/123580 (InhibRx)), each comprising different antigen binding sites but having, in common, the ability to compete with endogenous SIRPα for binding to CD47, thereby to allow interaction with macrophages and, ultimately, to increase the rate of CD47+ cancer cell depletion. These CD47 antibodies have activities in vivo that are quite different from those intrinsic to SIRPα-based agents. The latter, for instance, display negligible binding to red blood cells whereas the opposite property in CD47 antibodies creates a need for strategies that accommodate the agent “sink” that follows administration.

Still other agents are proposed for use in blocking the CD47/SIRPα axis. These include CD47Fc proteins (see Viral Logic's WO2010/083253), and SIRPα antibodies as described in UHN's WO2013/056352), and CD47 antibodies as described in Stanford's WO2016/022971 and many other patent publications.

The CD47 blockade approach in anti-cancer agent development shows great promise. It would be useful to provide methods and means for improving the effect of these agents, and in particular for improving the effect of the CD47 blocking agents, especially those that incorporate SIRPα.

### SUMMARY

It is now shown that the anti-cancer effect of a CD47-blocking form of SIRPαFc can be improved when combined with an agent that binds vascular endothelial growth factor (an anti-VEGF agent), such as the anti-VEGF antibody bevacizumab. More particularly, significant improvement in cancer cell depletion is seen when CD47+ cancer cells are treated with a CD47-blocking form of SIRPαFc, in combination with an anti-VEGF agent. Unexpectedly, the anti-cancer effect of the combination is seen even when each agent is used at a dose which, when used as a monotherapy, is too low to elicit anti-cancer effect against the type of cancer in question.

In one aspect, there is provided a method for treating a subject presenting with CD47+ disease cells, including CD47+ cancer cells, comprising administering to the subject a treatment-effective combination comprising (1) a CD47-binding form of SIRPαFc, wherein the SIRPα component comprises the SIRPαFc designated TTI-621 or the SIRPαFc designated TTI-622, and (2) an anti-VEGF agent such as an antibody, for instance, bevacizumab.

In a related aspect, there is provided the use of a CD47-binding form of SIRPαFc in combination with an anti-VEGF agent for the treatment of a subject presenting with CD47+ cancer, including lung cancer.

There is also provided, in another aspect, a combination of anti-cancer agents comprising a SIRPαFc-based CD47 blocking agent and an anti-VEGF agent, together with instructions teaching their use in the treatment method herein described.

To the extent that embodiments, details, or variations are described herein with reference to one particular SIRPα-based drug or one particular anti-VEGF agent, it should be understood that the same embodiments, details, and variations are intended to apply to others identified herein, unless this document or context explicitly indicates otherwise.

Various details and aspects are described herein as treating or methods of treating. In all such circumstances, it should be understood that related or equivalent aspects include the materials/compositions described herein for use in treatment; and the materials/compositions described herein for use in the manufacture of medicaments for treatment of diseases or conditions described herein.

The headings herein are for the convenience of the reader and not intended to be limiting. Other aspects of the invention will be apparent from the detailed description and claims that follow.

Additional embodiments of the invention are summarized in the following numbered embodiments (E):
E1. A method for treating a subject presenting with CD47+ disease cells, comprising administering to the subject a treatment-effective combination comprising (1) a CD47-binding form of SIRPαFc, and (2) an anti-VEGF agent.
E2. A method for improving the treatment of a subject presenting with CD47+ disease cells, said subject being treated with a CD47-binding form of SIRPαFc, the method comprising administering to the subject an anti-VEGF agent.
E3. A method for improving the treatment of a subject presenting with CD47+ disease cells, said subject being treated with an anti-VEGF agent, the method comprising administering to the subject a CD47-binding form of SIRPαFc.
E4. A CD47-binding form of SIRPαFc and an anti-VEGF agent for use in combination to treat a subject with CD47+ disease cells.
E5. An anti-VEGF agent for use in combination with a CD47-binding form of SIRPαFc to treat a subject presenting with CD47+ disease cells.
E6. Use of a CD47-binding form of SIRPαFc in the manufacture of a medicament for use in combination with an anti-VEGF agent for the treatment of CD47+ disease cells in a subject.
E7. Use of an anti-VEGF agent in the manufacture of a medicament for use in combination with a CD47-binding form of SIRPαFc for the treatment of CD47+ disease cells in a subject.
E8. The method, use, or drug-for-use according to any one of E1-E7, wherein the anti-VEGF agent is an anti-VEGF antibody or an anti-VEGF receptor antibody.
E9. The method, use, or drug-for-use according to E8, wherein the anti-VEGF agent is an anti-VEGF antibody.
E10. The method, use, or drug-for-use according to E9, wherein the anti-VEGF agent is bevacizumab or a VEGF-binding fragment or variant of bevacizumab.
E11. The method, use, or drug-for-use according to E9 or E10, wherein the anti-VEGF agent is a VEGF-A binding antibody or VEGF-A-binding fragment thereof comprising the CDR sequences of bevacizumab.
E12. The method, use, or drug-for-use according to E10, wherein the anti-VEGF agent is Avastin®.
E13. The method, use, or drug-for-use according to E8, wherein the anti-VEGF agent is an anti-VEGF receptor antibody.
E14. The method, use, or drug-for-use according to E13, wherein the anti-VEGF agent is an anti-VEGFR-2 antibody.
E15. The method, use, or drug-for-use according to E13, wherein the anti-VEGF agent is ramucirumab, or a VEGFR-2 binding fragment of ramucirumab, or an antibody or fragment thereof that comprises the CDR sequences of ramucirumab.
E16. The method, use, or drug-for-use according to E13, wherein the anti-VEGF receptor antibody is ramucirumab.
E17. The method, use, or drug-for-use according to any one of E1-E16, wherein the SIRPαFc comprises the IgV region of human SIRPα variant 2.
E18. The method, use, or drug-for-use according to E17, wherein the SIRPαFc fusion protein comprises the amino acid sequence of SEQ ID NO: 3.
E19. The method, use, or drug-for-use according to E17, wherein the SIRPαFc fusion protein comprising comprises the amino acid sequence of SEQ ID NO: 8.
E20. The method, use, or drug-for-use according to any one of E1-E19, wherein the CD47+ disease cells comprise CD47+ cancer cells.
E21. The method, use, or drug-for-use according to E20, wherein the CD47+ cancer cells are blood cancer cells or solid tumour cells.
E22. The method, use, or drug-for-use according to E10, wherein the CD47+ cancer cells comprise blood cancer cells.
E23. The method, use, or drug-for-use according to E22, wherein the blood cancer cells comprise leukemia cells.
E24. The method, use, or drug-for-use according to E23, wherein the leukemia is acute lymphocytic leukemia (ALL); acute myeloid leukemia (AML); chronic lymphocytic leukemia (CLL); chronic myelogenous leukemia (CML); myeloproliferative disorder/neoplasm (MPDS); or myelodysplastic syndrome (MDS).
E25. The method, use, or drug-for-use according to E22, wherein the blood cancer cells comprise lymphoma cells.
E26. The method, use, or drug-for-use according to E25, wherein the lymphoma is a T cell lymphoma, peripheral T cell lymphoma (PTCL), cutaneous T cell lymphoma (CTCL), Hodgkin's lymphoma, indolent non-Hodgkin's lymphoma, aggressive non-Hodgkin's lymphoma, Burkitt's lymphoma, small cell and follicular lymphoma, large cell follicular lymphoma, mycosis fungoides, Sezary syndrome, primary cutaneous T cell lymphoproliferative (PCTL) disorders, or anaplastic large cell lymphoma.
E27. The method, use, or drug-for-use according to E22, wherein the blood cancer is a myeloma.
E28. The method, use, or drug-for-use according to E27, wherein the myeloma is multiple myeloma (MM), giant cell myeloma, heavy-chain myeloma, or light chain or Bence-Jones myeloma.
E29. The method, use, or drug-for-use according to E21, wherein the cancer cells comprise solid tumour cells.
E30. The method, use, or drug-for-use according to E29, wherein the solid tumour cancer cells comprise breast cancer cells.
E31. The method, use, or drug-for-use according to E29, where in the solid tumour cancer cells comprise lung cancer cells.
E32. The method, use, or drug-for-use according to E29, wherein the solid tumour cancer cells comprise ovarian cancer cells.
E33. The method, use, or drug-for-use according to E29, wherein the solid tumour CD47+ cancer cells are selected from colorectal, renal, hepatocellular carcinoma and glioblastoma cells.
E34. The method, use, or drug-for-use according to any one of E1-E19, wherein the subject undergoing treatment has a disease selected from among the group consisting of Crohn's disease, allergic asthma, rheumatoid arthritis, age-related macular degeneration, AMD, diabetic retinopathy, liver fibrosis and angiosarcoma.
E35. A combination of anti-cancer agents, comprising an amount of a CD47 binding form of SIRPαFc, and an amount of bevacizumab effective, in combination, to deplete CD47+ cancer cells, together with instructions teaching the use thereof according to any one of E1-E12 and E17-E34.
E36. The use of the combination according to E35, for the treatment of a subject presenting with CD47+ disease cells.
E37. The use according to E36, wherein the CD47+ disease cells are CD47+ cancer cells.
E38. The use according to E37, wherein the cancer cells are cells of a cancer type selected from acute lymphocytic leukemia (ALL); acute myeloid leukemia (AML); p53-mutated AML, chronic lymphocytic leukemia (CLL); chronic myelogenous leukemia (CML); myeloproliferative disorder/neoplasm (MPDS); and myelodysplastic syndrome.
E39. The use according to E37, wherein the cancer is a lymphoma selected from a Hodgkin's lymphoma, indolent non-Hodgkin's lymphoma, aggressive non-Hodgkin's lymphoma, Burkitt's lymphoma, small cell follicular lymphoma, large cell follicular lymphoma, and diffuse large B cell lymphoma (DLBCL).
E40. The use according to E37, wherein the cancer is a myeloma selected from multiple myeloma (MM), giant cell myeloma, heavy-chain myeloma, and light chain or Bence-Jones myeloma.
E41. The use according to any one of E36-E40, wherein the CD47-blocking from of SIRPαFc comprises SEQ ID NO: 3.
E42. The use according to any one of E36-E40, wherein the CD47-blocking from of SIRPαFc comprises SEQ ID NO: 8.
E43. A kit comprising unit dose formulations of a CD47-binding form of SIRPαFc, and (2) an anti-VEGF agent.
E44. The kit according to E43, wherein the CD47-binding form of SIRPαFc, and (2) an anti-VEGF agent are packaged together but not in admixture.

These and other aspects of the invention are now described in greater detail with reference to the accompanying drawings, in which:

### BRIEF REFERENCE TO THE DRAWINGS

FIG. 1A and FIG. 1B show that the combination of SIRPαFc-based agents and an anti-VEGF agent induces tumor control in the NCI-H358 lung cancer xenograft model.

FIG. 2A and FIG. 2B show that the combination of SIRPαFc-based agents and an anti-VEGF agent increases survival in the NCI-H358 lung cancer xenograft model.

### DETAILED DESCRIPTION

The present invention provides an improved method and combination for treating subjects that present with cancer cells and tumours that have a CD47+ phenotype. In this method, subjects receive a combination of a CD47 blocking agent that is a CD47-binding form of SIRPα, and an anti-VEGF agent. In combination, the anti-cancer effect is superior to the effects of either agent alone.

Thus, the present treatment method combines a CD47-binding form of SIRPα, as a CD47 blocking agent, and an anti-VEGF agent.

A CD47 blocking agent is defined herein as a CD47-binding agent that interferes with and dampens or inhibits signal transmission that results when CD47 interacts with macrophage-presented SIRPα. CD47-binding forms of human SIRPα are the preferred CD47 blocking agents for use in the combination herein disclosed. These agents are based on the extracellular region of human SIRPα. They comprise at least a part of the SIRPα extracellular region sufficient to confer effective CD47 binding affinity and specificity. Forms of SIRPα, lacking the membrane anchoring and intracellular components, are described in the literature and include those referenced in WO2010/130053 (University Health Network), WO 2010/070047 (Novartis), WO2013/109752 (Stanford), EP3180363 (Merck), and WO2014/094122 (Trillium). Each of these documents is incorporated by reference in its entirety and for the specific disclosures relating to SIRPα-based constructs.

Unless otherwise stated, the term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 50-80% of the forms of SIRPα present in humans, and both are embraced herein by the term “human SIRPα”. Also embraced by the term “human SIRPα” are the minor forms thereof that are endogenous to humans and have the same property of triggering signal transduction through CD47 upon binding thereto. The present invention is directed most particularly to the agent combinations that include the human SIRP variant 2 form, or V2, and especially the IgV domain thereof (known also as the d1 region).

In the present agent combination, useful CD47-binding SIRPαFc fusion proteins comprise one of the three so-called immunoglobulin (Ig) domains that lie within the extracellular region of human SIRPα. More particularly, the present SIRPαFc proteins incorporate residues 32-137 of human SIRPα (a 106-mer), which constitute and define the IgV domain of the V2 form according to current nomenclature. This SIRPα sequence, shown below, is referenced herein as SEQ ID NO: 1:

(SEQ ID NO: 1)
EELQVIQPDKSVSVAAGESAILHCTVTSLIPVGPIQWFRGAGPARELIYN
QKEGHFPRVTTVSESTKRENMDFSISISNITPADAGTYYCVKFRKGSPDT
EFKSGA

In a preferred embodiment, the SIRPαFc fusion proteins incorporate the IgV domain as defined by SEQ ID NO: 1, and additional, flanking residues contiguous within the SIRPα sequence. This preferred form of the IgV domain, represented by residues 31-148 of the V2 form of human SIRPα, is a 118-mer having SEQ ID NO: 5 shown below:

(SEQ ID NO: 5)
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPS

In a preferred embodiment, the CD47-binding form of SIRPα is an Fc fusion. More particularly, the agent suitably comprises a CD47-binding fragment of human SIRPα protein, in a form fused directly, or indirectly, with an antibody constant region, or Fc region, having at least some effector function. Fc refers to “fragment crystallisable” and represents the constant region of an antibody comprised principally of the heavy chain constant region and components within the hinge region. An Fc component “having effector function” is an Fc component having at least some contribution for instance to antibody-dependent cellular cytotoxicity or some ability to fix complement. Also, the Fc will at least bind to an Fc receptor. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Val234, Ala235 and deletion of Gly236 (EU), is considered not to have effector function. In a preferred embodiment, the Fc is based on human antibodies of the IgG1 isotype. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID NO: 2:

(SEQ ID NO: 2)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK*

Thus, in embodiments, the Fc region has either a wild type or consensus sequence of an IgG1 constant region. In alternative embodiments, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469, all incorporated herein by reference.

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In a specific embodiment, the Fc region is based on the amino acid sequence of a human IgG4 set out as P01861 in UniProtKB/Swiss-Prot, residues 99-327, and has the amino acid sequence shown below and referenced herein as SEQ ID NO: 6:

(SEQ ID NO: 6)
ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGOPENNYKTTPPVLDSDGSFFLYSRLTVDKSRWQ
EGNVFSCSVMHEALHNHYTQKSLSLSLGK

In embodiments, the Fc region incorporates one or more alterations, usually not more than about 10, e.g., up to 5 such alterations, including amino acid substitutions that affect certain Fc properties. In one specific and preferred embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F as taught in U.S. 62/777,375, and many others. Particularly useful are those alterations that enhance Fc properties while remaining silent with respect to conformation, e.g., retaining Fc receptor binding.

In a specific embodiment, and in the case where the Fc component is an IgG4 Fc, the Fc incorporates at least the S²²⁸P mutation, and has the amino acid sequence set out below and referenced herein as SEQ ID NO: 7:

(SEQ ID NO: 7)
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGOPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

The CD47 blocking agent used in the combination is thus preferably a SIRP fusion protein useful to inhibit the binding of human SIRPα and human CD47, thereby to inhibit or reduce transmission of the signal mediated via SIRPα-bound CD47, the fusion protein comprising a human SIRPα component and, fused therewith, an Fc component, wherein the SIRPα component comprises or consists of a single IgV domain of human SIRPα V2 and the Fc component is the constant region of a human IgG having effector function.

In one embodiment, the fusion protein comprises a SIRPα component consisting at least of residues 32-137 of the V2 form of wild type human SIRPα, i.e., SEQ ID NO: 1. In a preferred embodiment, the SIRPα component consists of residues 31-148 of the V2 form of human SIRPα, i.e., SEQ ID NO: 5. In another embodiment, the Fc component is the Fc component of the human IgG1 designated P01857, and in a specific embodiment has the amino acid sequence that incorporates the lower hinge-CH2-CH3 region thereof, i.e., SEQ ID NO: 2.

In a preferred embodiment, therefore, the SIRPαFc fusion protein is provided and used in a secreted dimeric fusion form, wherein the fusion protein incorporates a SIRPα component having SEQ ID NO: 1 and preferably SEQ ID NO: 5 and, fused therewith, an Fc region having effector function and having SEQ ID NO: 2 or SEQ ID NO: SEQ ID NO: 7.

When the SIRPα component is SEQ ID NO: 5 and fused therewith an Fc region having effector function and having SEQ ID NO: 2, this fusion protein comprises SEQ ID NO: 3, shown below:

(SEQ ID NO: 3)
EEELQVIQPDKSVSVAAGESAILHCTVTSLIPVGPIQWFRGAGPARELIY
NQKEGHFPRVITVSESTKRENMDFSISISNITPADAGTYYCVKFRKGSPD
TEFKSGAGTELSVRAKPSDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK

In alternative embodiments, the Fc component of the fusion protein is based on an IgG4, and preferably an IgG4 that incorporates the S²²⁸P mutation. In the case where the fusion protein incorporates the preferred SIRPα IgV domain of SEQ ID NO: 5, the resulting IgG4-based SIRPα-Fc protein has SEQ ID NO: 8, shown below:

(SEQ ID NO: 8)
EEELQVIQPDKSVSVAAGESAILHCTVTSLIPVGPIQWERGAGPARELIY
NQKEGHFPRVTTVSESTKRENMDFSISISNITPADAGTYYCVKFRKGSPD
TEFKSGAGTELSVRAKPSESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK

In preferred embodiment, the fusion protein comprises, as the SIRPα IgV domain of the fusion protein, a sequence that is or comprises SEQ ID NO: 5. The preferred SIRPαFc comprises or is SEQ ID NO: 3 (TTI-621). In another further preferred embodiment, the SIRPαFc is SEQ ID NO: 8 (TTI-622).

The SIRPα sequence incorporated within the CD47 blocking agent can be varied, as described in the literature. That is, useful substitutions within SIRPα include one or more of the following: L⁴V/I, V⁶I/L, A²¹V, V²⁷I/L, I³¹T/S/F, Q³⁷W, Q³⁷H, E⁴⁷V/L, K⁵³R, E⁵⁴Q, E⁵⁴P, H⁵⁶P/R, S⁶⁶T/G, K⁶⁸R, M⁷²R, V⁹²I, F⁹⁴V/L, V⁶³I, and/or F¹⁰³V. (Amino acid position numbers correspond with sequences shown herein, including SEQ ID NO: 5). Substitutions that remove glycosylation sites are also acceptable as are additions and/or deletions especially of terminal amino acids such as residues 1 or 1 and 2, and C-terminal residues 1, 2, 3, 4, or 5. Suitable variants will display adequate CD47-binding activity and CD47 antagonist activity, with respect to signal transmission between CD47 and SIRPα.

In the SIRPαFc fusion protein, the SIRPα component and the Fc component are fused, either directly or indirectly, to provide a single chain polypeptide that is ultimately produced as a dimer in which the single chain polypeptides are coupled through intrachain disulfide bonds formed within the Fc region. The nature of the fusing region is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, ClaI, EcoRI, HindIII, PstI, SalI and XhoI site and the like.

The linker amino acids typically and desirably have some flexibility to allow the Fc and the SIRPα components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G₄S sequence (Gly-Gly-Gly-Gly-Ser) (SEQ ID NO: 9) which may repeat as (G₄S)ₙ where n is 1, 2, 3 or more, or is based on (Gly)n, (Ser)n, (Ser-Gly)n or (Gly-Ser)n and the like. In another embodiment, the linker is GTELSVRAKPS (SEQ ID NO: 4). This sequence constitutes SIRPα sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

The SIRPαFc can, as a single chain polypeptide, be fused with a different Fc fusion protein to provide an Fc dimer that is bispecific in its affinity. For instance, SIRPαFc can be coupled with an Fc fusion or antibody that binds a tumour-specific antigen, such as epidermal growth factor receptor (EGFR) and other target cancer cell antigens. Bispecific proteins can also be generated by coupling/fusing a protein of medical interest to the N- or C-terminus of SIRPα or SIRPαFc.

As noted, the SIRPαFc fusion is useful to inhibit interaction between SIRPα and CD47, thereby to block signalling across this axis. Stimulation of SIRPα on macrophages by CD47 is known to inhibit macrophage-mediated phagocytosis by deactivating myosin-II and the contractile cytoskeletal activity involved in pulling a target into a macrophage. Activation of this cascade is therefore important for the survival of CD47+ disease cells, and blocking this pathway enables macrophages to eradicate/deplete or at least reduce the vitality, the number, or the distribution, for instance, of the CD47+ cancer cell population.

The term “CD47+” (or the equivalent CD47+) is used with reference to the phenotype of cells targeted for binding by the present polypeptide agents. Cells that are CD47+ can be identified by flow cytometry using CD47 antibody as the affinity ligand. CD47 antibodies that are labeled appropriately are available commercially for this use (for example, the antibody product of clone B6H12 is available from Santa Cruz Biotechnology). The cells examined for CD47 phenotype can include standard tumour biopsy samples including particularly blood samples taken from the subject suspected of harbouring endogenous CD47+ cancer cells. CD47 disease cells of particular interest as targets for therapy with the present fusion proteins are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

The present agent combination comprises both a CD47-binding form of SIRPα, as just described, and an anti-VEGF agent. VEGF (vascular endothelial growth factor) is a protein involved in angiogenesis and includes VEGF-A, a human protein having the amino acid sequence reported in UniProt as P15692 as well as its various isoforms. VEGF, and particularly VEGF-A, is a key cytokine in the development of normal blood vessels as well as the development of vessels in tumors and other tissues undergoing abnormal angiogenesis. “Anti-VEGF” agents include agents that bind circulating VEGF-A and thereby inhibit interaction with VEGF receptors (VEGFR-1, VEGFR-2, etc.) the signalling of which is involved in tumour angiogenesis. Circulating VEGF binds to VEGF receptors VEGFR-1 and VEGFR-2 and to its coreceptors neuropilin (NRP)-1 and NRP-2 with high binding affinity. These receptors are expressed on the surface of endothelial cells, and they play a critical role in the development of angiogenesis by stimulating the recruitment and proliferation of endothelial cells.

Bevacizumab is a humanized anti-VEGF monoclonal IgG1 antibody (now sold under the mark Avastin®), and acts by selectively binding up to 97% of circulating VEGF, thereby inhibiting the binding of VEGF to its cell surface receptors, VEGFR-1 and VEGFR-2. This inhibition leads to a reduction in microvascular growth of tumor blood vessels and thus limits the blood supply to tumor tissues. These effects also lower tissue interstitial pressure, increase vascular permeability, and favor apoptosis of tumor endothelial cells.

Bevacizumab is reported to have the heavy and light chain amino acid sequences shown below:

Heavy chain:
(SEQ ID NO: 10)
MEFGLSWLFL VAILKGVQCE VQLVESGGGL VQPGGSLRLS
CAASGYTFTN YGMNWVRQAP GKGLEWVGWI NTYTGEPTYA
ADFKRRFTFS LDTSKSTAYL QMNSLRAEDT AVYYCAKYPH
YYGSSHWYFD VWGQGTLVTV SSASTKGPSV FPLAPSSKST
SGGTAALGCL VKDYFPEPVT VSWNSGALTS GVHTFPAVLQ
SSGLYSLSSV VTVPSSSLGT QTYICNVNHK PSNTKVDKKV
EPKSCDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR EEMTKNQVSL TCLVKGFYPS
DIAVEWESNG QPENNYKTTP PVLDSDGSFF LYSKLTVDKS
RWQQGNVFSC SVMHEALHNH YTQKSLSLSP GKKDEL
Light chain:
(SEQ ID NO: 11)
MKYLLPTAAA GLLLLAAQPA MADIQMTQSP SSLSASVGDR
VTITCSASQD ISNYLNWYQQ KPGKAPKVLI YFTSSLHSGV
PSRFSGSGSG TDFTLTISSL QPEDFATYYC QQYSTVPWTF
GQGTKVEIKR TVAAPSVFIF PPSDEQLKSG TASVVCLLNN
FYPREAKVQW KVDNALQSGN SQESVTEQDS KDSTYSLSST
LTLSKADYEK HKVYACEVTH QGLSSPVTKS FNRGECKDEL

This antibody can be characterized by its complementarity determining regions (CDRs) which, together with the intervening framework regions, provide the antibody with its anti-VEGF binding and bioactivity. Thus, antibodies and antigen-binding fragments of bevacizumab are useful herein as anti-VEGF agents, including antibodies or antigen binding fragments of bevacizumab that comprise the heavy and light chain CDRs having exemplary amino acid sequences noted below:

Heavy Chain:
(SEQ ID NO: 12)
CDR1: GYTFTNYGMN
(SEQ ID NO: 13)
CDR2: WINTYTGEPTYAADFKR
(SEQ ID NO: 14)
CDR3: YPHYYGSSHWYFDV
Light chain:
(SEQ ID NO: 15)
CDR1: SASQDISNYLN
(SEQ ID NO: 16)
CDR2: FTSSLHS
(SEQ ID NO: 17)
CDR3: QQYSTVPWT

These sequences are sourced from FIG. 27 of AbbVie's U.S. Pat. No. 9,815,893, incorporated herein by reference. Bevacizumab is approved for the treatment of advanced colorectal cancer (CRC), advanced non-small cell lung cancer (NSCLC), metastatic breast cancer (MBC) and advanced renal cell cancer. As a single agent, it has been approved for second-line treatment of advanced glioblastoma multiforme. Further studies are being conducted in other solid tumors as well.

Bevacizumab is a clear, colorless solution administered i.v. in doses of up to about 20 mg/kgs, such as 5, 7.5, 10 or 15 mg/kg. When used in combination with SIRPαFc, the bevacizumab dose can be at the lower end of the useful scale, e.g., at about 0.5 mg/kg.

Alternative anti-VEGF agents include other VEGF antibodies that bind VEGF, such as VEGF-A, and block VEGF binding with VEGFR, as well as VEGFR antibodies that block the target receptor from binding with VEGF. Specific examples useful as anti-VEGF agents in embodiments of the present method include ranibizumab, an affinity-matured antibody Fab domain approved for use in age-related macular degeneration, and IMC-18F1, a monoclonal antibody that target the VEGF receptors VEGFR-2 and VEGFR-1, respectively. Aflibercept (a VEGF-Trap), is useful as a peptide-antibody fusion that targets VEGF ligand.

In a specific embodiment the anti-VEGF agent is Avastin®, a humanized anti-VEGF monoclonal IgG1 antibody formulated for administration by infusion or by injection. In combination with chemotherapy, it is approved for the treatment of advanced colorectal cancer (CRC), advanced non-small cell lung cancer (NSCLC), metastatic breast cancer (MBC), epithelial ovarian, fallopian tube or primary peritoneal cancer and advanced renal cell cancer. It is also approved in combination with atezolizumab in hepatocellular carcinoma (HCC). As a single agent, it has been approved for second-line treatment of advanced glioblastoma multiforme.

Bevacizumab can be used in combination with SIRPαFc in the treatment of cancers such as advanced colorectal cancer (CRC), advanced non-small cell lung cancer (NSCLC), metastatic breast cancer (MBC) and advanced renal cell cancer. As a single agent, it has been approved for second-line treatment of advanced glioblastoma multiforme. In embodiments the present combination is used to treat ovarian cancer, and the drug is being used at a dose of 10 or 15 mg/kg every 3 weeks for instance in combination with carboplatin, carboplatin with gemcitabine, topotecan, or pegylated liposomal doxorubicin.

Each agent included in the combination can be formulated separately for use in combination. The agents are said to be used “in combination” when, in a recipient of both agents, the effect of one agent enhances the effect of the other.

In this approach, each agent is provided in a dosage form comprising a pharmaceutically acceptable carrier, and in a therapeutically effective amount. As used herein, “pharmaceutically acceptable carrier” means any and all solvents, dispersion media, coatings, antibacterial and antifungal agents, isotonic and absorption delaying agents, and the like that are physiologically compatible and useful in the art of protein/antibody formulation. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. Each of the SIRPαFc fusion protein and the anti-VEGF agent is formulated using practises standard in the art of therapeutic protein formulation. Solutions that are suitable for intravenous administration, such as by injection or infusion, are particularly useful.

As used herein, “effective amount” and “treatment-effective” amounts refer to amounts effective, in combination, at dosages and for a particular period of time necessary, to achieve the desired therapeutic result. A treatment-effective amount of each agent in the combination may vary according to factors such as the disease state, age, sex, and weight of the individual, and the ability of the agent to elicit a desired response in the recipient. As is exemplified herein, the amount of a single one of the agents in the combination can be an amount that is too small to elicit a treatment-effective response on its own. However, when that same amount is used in combination with the second agent as in the present method, a treatment-effect response can result even when no further amount of the first agent is added. In other words, the SIRPαFc agents, including TTI-621 and TTI-622 can enhance the effect of an ineffective dose of the anti-VEGF agent that is Avastin®. Alternatively, the anti-VEGF agents including bevacizumab and its Fab fragments, can enhance the effect of an ineffective dose of the SIRPαFc agent including TTI-621 and TTI-622. A treatment effective amount is also one in which any toxic or detrimental effects of the pharmacological agent are outweighed by the therapeutically beneficial effects. Treatment-effective dosing will elicit one or more end-points that include enhanced patient survival, reduction in tumour burden including reduced tumour size, stunted growth rate, reduced cancer vitality and limited distribution and metastasis, for instance.

The anti-VEGF agent will of course be formulated in amounts that are suitable for patient dosing, as permitted by the regulatory agencies that have approved its use in humans. For bevacizumab, effective doses will include 2-4 milligrams given intravenously such as by infusion over the course of 5-15 minutes, for instance. Elevated levels of circulating VEGF have been associated with a poor prognosis and greater risk for the development of metastases in patients with different cancer types. There are many techniques used to evaluate VEGF expression in human tumors, including immunohistochemistry, ELISA, chemiluminescence immunosorbent assay, Western blotting, and in situ hybridization

The SIRPαFc fusion protein can be administered to the subject through any of the routes established for protein delivery, in particular intravenous, intradermal and subcutaneous injection or infusion, intratumoural injection, or by oral or nasal or pulmonary administration.

The agents in the present combination can be administered, as discrete agents, sequentially or essentially at the same time. In embodiments, the anti-VEGF agent is given before administration of SIRPαFc. It is not essential that the anti-VEGF agent is present in a patient's system when the CD47 blocking agent is administered, although this is suitable. Thus, in one embodiment there is provided a method for treating a subject presenting with CD47+ disease cells, comprising administering bevacizumab to the subject and then administering SIRPαFc to that subject in amounts sufficient to reduce the CD47+ disease/cancer cell population. In another embodiments, the SIRPαFc is administered before the anti-VEGF agent is given. Desirably, the subject is treated such that the effect/s of one agent is present in the subject when the other agent is administered. Thus, an anti-VEGF agent can be administered to a subject that has been treated with SIRPαFc, and SIRPαFc can be administered to a subject that has been treated with an anti-VEGF agent.

Dosing regimens are adjusted to provide the optimum desired response (e.g., a therapeutic response). For example, a single bolus of each agent may be administered, or several divided doses may be administered over time or the dose may be proportionally reduced or increased as indicated by the therapeutic situation. It is especially advantageous to formulate parenteral compositions in unit dosage form for ease of administration and uniformity of dosage. “Unit dosage form” as used herein refers to physically discrete units suited as unitary dosages for the subjects to be treated; each unit contains a predetermined quantity of active compound calculated to produce the desired therapeutic effect in association with the required pharmaceutical carrier.

The agents can be formulated in combination, so that the combination can be introduced to the recipient in one administration, e.g., one injection or one infusion. Alternatively, the agents can be combined as separate units that are provided together in a single package, and with instructions for the use thereof according to the present method. In another embodiment, an article of manufacture containing the SIRPαFc agent and anti-VEGF agent combination in an amount useful for the treatment of the disorders described herein is provided. The article of manufacture comprises one or both agents of the present antibody agent combination, as well as a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with the container can indicate that the composition is to be used in a combination of anti-VEGF agent with SIRPαFc, in accordance with the present invention, thereby to elicit a co-operative effect on the CD47+ disease cells. The article of manufacture may further comprise a second container comprising a pharmaceutically-acceptable buffer, such as phosphate-buffered saline, Ringer's solution and dextrose solution. It may further include other matters desirable from a commercial and use standpoint, including other buffers, diluents, filters, needles, syringes, and package inserts with instructions for use.

For administration, with either SIRPαFc of SEQ ID NO: 3 (TTI-621) or of SEQ ID NO: 8 (TTI-622), the suitable SIRPαFc dose will be within the range from about 0.0001 to 100 mg/kg, and more usually 0.01 to 50 mg/kg, of the patient body weight. For example, dosages can be 0.3 mg/kg body weight, 1 mg/kg body weight, 3 mg/kg body weight, 5 mg/kg body weight or 10 mg/kg body weight, 20 mg/kg body weight, or within the range of 0.1-25 mg/kg.

The SIRPαFc protein displays negligible binding to red blood cells, relative to most CD47 antibodies. There is accordingly no need to account for an RBC “sink” when dosing with the agent combination. Relative to the other CD47 blocking agents that are bound by RBCs, it is estimated that the present SIRPαFc fusion can be effective at doses that are less than half the doses required for agents that become RBC-bound, such as CD47 antibodies. Moreover, the SIRPα-Fc fusion protein is a dedicated antagonist of the SIRPα-mediated signal, as it displays negligible CD47 agonism. There is accordingly no need, when establishing medically useful unit dosing regimens, to account for any CD47 stimulation induced by the agent.

The agent combination is useful to treat a variety of CD47+ disease cells. These include particularly CD47+ cancer cells, including liquid and solid tumours. Solid tumours can be treated with the present agent combination, to reduce the size, number or growth rate thereof and to control growth of cancer stem cells. Such solid tumours include CD47+ tumours in bladder, brain, breast, lung, colon/colorectal, ovary, pancreas, prostate, liver, kidney, renal, skin and other tissues as well. In another embodiment, the agent combination can used to inhibit the growth or proliferation of hematological cancers. As used herein, “hematological cancer” refers to a cancer of the blood, and includes leukemia, lymphoma and myeloma among others.

“Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML) including P53-mutated AML; chronic lymphocytic leukemia (CLL); chronic myelogenous leukemia (CML); myeloproliferative disorder/neoplasm(MPDS); and myelodysplastic syndrome. In some embodiments, the hematological cancer treated with the agent combination is a CD47+ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

“Lymphoma” includes T cell lymphomas, and may refer to a Hodgkin's lymphoma, both indolent and aggressive non-Hodgkin's lymphoma, Burkitt's lymphoma, and follicular lymphoma (small cell and large cell), diffuse large B cell lymphoma (DLBCL), peripheral T cell lymphoma including PTCL, and cutaneous T cell lymphoma CTCL, among others. In particular embodiments, the combination is used to treat T cell lymphomas that are a very heterogeneous group of lymphoid malignancies divided into cutaneous and peripheral TCL, which themselves are divided into nodal or extranodal types. CTCL derives from skin-homing T cells and consists of mycosis fungoides, Sezary syndrome, primary cutaneous T cell lymphoproliferative disorders, and anaplastic large cell lymphoma. The common features of TCL are aggressive course and poor response to therapy.

“Myeloma” may refer to multiple myeloma (MM), giant cell myeloma, heavy-chain myeloma, and light chain or Bence-Jones myeloma.

In other embodiments, the hematological cancer treated with the agent combination is a CD47+ lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma.

Solid CD47+ tumours including sarcomas and carcinomas and adenocarcinoma, as well as angiosarcoma, can also be treated with the present combination method, including leiomyosarcoma, and solid cancers of various tissues including lung, breast, colon, prostate, ovary, testicle, prostate, bladder, pancreas, liver, brain, kidney, etc. Conditions and diseases treatable with the combination can also include those responsive to anti-VEGF agent per se including but not limited to Crohn's disease, allergic asthma, rheumatoid arthritis, age-related macular degeneration, diabetic retinopathy, and fibrosis including liver fibrosis.

The combination therapy, comprising a CD47 blocking form of SIRPαFc and anti-VEGF agent-mediated treatment, can also be exploited together with any other agent or modality useful in the treatment of the targeted indication, such as surgery as in adjuvant therapy, or with additional chemotherapy as in neoadjuvant therapy.

Incorporated by reference herein for all purpose is the content of U.S. Provisional Patent Application No. 63/277,101 (filed Nov. 8, 2021).

### EXAMPLES

### Example 1: Combination of TTI-622 or TTI-621 and Anti-VEGF Agent Induces Tumor Growth Control

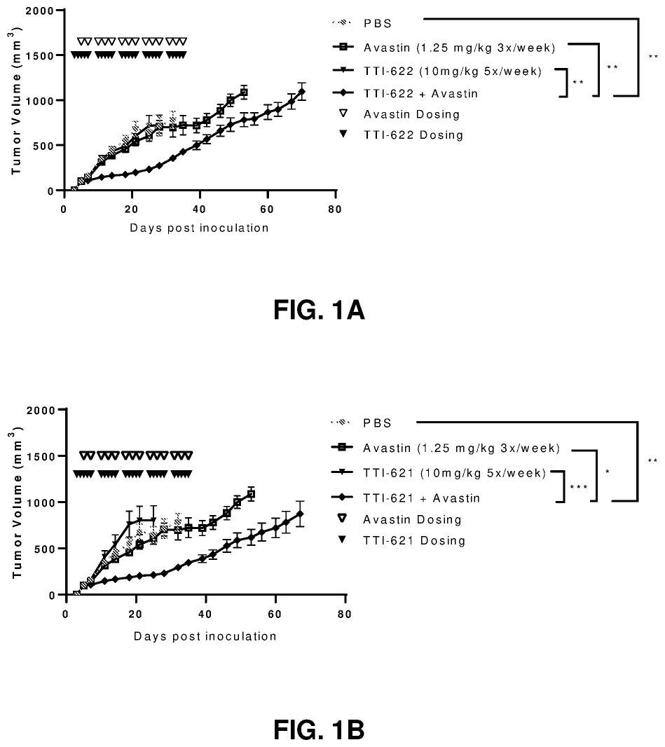

1×10\hat{ }7 NCI-H358 (ATCC CRL-5807) non-small cell lung cancer cells were implanted into NOD-SCID mice. On day 3 after implantation, mice were randomized into groups dosed with PBS or either (A) TTI-622 or (B) TTI-621 at 10 mg/kg. Tumor growth was monitored daily until the tumor volume in the mice dosed with PBS reached between 80-150 mm\hat{ }3; this occurred on day 5 after implantation. The mice were then further randomized to receive Avastin® at 1.25 mg/kg, three times per week alone, or in combination with TTI-622 (A) or TTI-621 (B). There was also a control group who were not dosed with Avastin® or TTI-621 or TTI-622. Mice were treated with the respective different single agents or combinations of Avastin® and TTI-621/TTI-622 for 5 weeks. Tumor growth was monitored twice a week and tumor volume was calculated in mm′using the formula: V=(L×W×W)/2, where V is tumor volume, L is tumor length (the longest tumor dimension) and W is tumor width (the longest tumor dimension perpendicular to L). One-way ANOVA was used to calculated differences in tumor growth between groups at day 28 for (A) TTI-622 and at day 25 for (B) TTI-621, *p<0.05, **p<0.01, ***p<0.001. As shown in FIG. 1A and FIG. 1B, when TTI-622 (A), TTI-621 (B) or Avastin were administered as single agent at the noted doses, there was no difference between tumor growth of the NCI-H538 tumor cells in these animals compared to animals treated with PBS control. However, when TTI-622 (A) or TTI-621 (B) were combined with Avastin® there was a significant decrease in tumor growth compared to the monotherapy arms or PBS control, demonstrating a synergistic effect of the combination of SIRPαFc and Avastin®.

In each of FIG. 1A and FIG. 1B, the data points in the graph for tumor volume from mice treated with the various agents are notated with shapes as follows: PBS: circle; Avastin®: square; TTI-622 (FIG. 1A) or TTI-621 (FIG. 1B): triangle; TTI-622+Avastin® (FIG. 1A) or TTI-621+Avastin® (FIG. 11B): diamond. The datapoints for tumor volume from mice treated with PBS and the individual agents of Avastin®, TTI-622, or TTI-621 largely overlap; the datapoints for TTI-622+Avastin® (FIG. 1A) or TTI-621+Avastin® (FIG. 1B), form a line that is distinct and furthest to the right in the graph from the other datapoints.

### Example 2: Combination of TTI-622 or TTI-621 and Anti-VEGF Agent Increases Survival

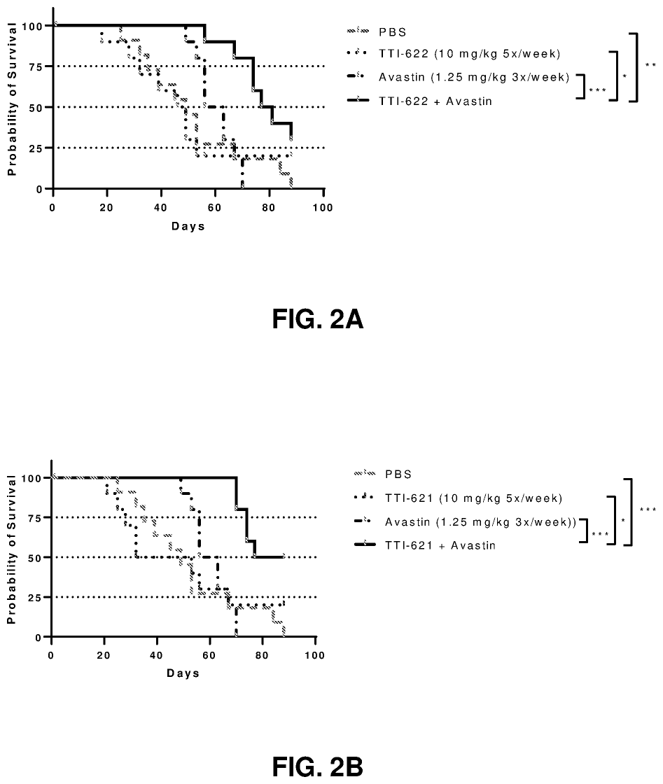

1×10\hat{ }7 NCI-H358 (ATCC CRL-5807) non-small cell lung cancer cells were implanted into NOD-SCID mice. On day 3 after implantation, mice were randomized into groups dosed with PBS or either (A) TTI-622 or (B) TTI-621 at 10 mg/kg. Tumor growth was monitored daily until the tumor volume in the mice dosed with PBS reached between 80-150 mm\hat{ }3; this occurred on day 5 after implantation. The mice were then further randomized to receive Avastin® at 1.25 mg/kg, three times per week alone, or in combination with TTI-622 (A) or TTI-621 (B). There was also a control group who were not dosed with Avastin® or TTI-621 or TTI-622. Mice were treated with the respective different single agents or combinations of Avastin® and TTI-621/TTI-622 for 5 weeks. Tumor growth was monitored twice a week and when tumor volume was equal or greater than 1500 mm\hat{ }3 the mice were considered to be sacrificed. Survival curves differences were analyzed by log-rank test, *p<0.05, **p<0.01, ***p<0.001. As shown in FIG. 2A and FIG. 2B, when TTI-622 (A), TTI-621 (B) or Avastin® were administered as single agent at the noted doses, there was no difference between survival of animals compared to animals treated with PBS control. However, when TTI-622 (A) or TTI-621 (B) were combined with Avastin® there was a significant increase in survival compared to the monotherapy arms or PBS control, demonstrating a synergistic effect of the combination of SIRPαFc and Avastin®. (In FIG. 2A, the TTI-622+Avastin® data is the line furthest on the right side; similarly, in FIG. 2B, the TTI-621+Avastin® data is the line furthest on the right side.)

While materials and methods of this disclosure have been described in terms of preferred embodiments, it will be apparent that variations may be applied to the articles and methods without departing from the spirit and scope of the disclosure. All such variations and equivalents, whether now existing or later developed, are deemed to be within the spirit and scope of the disclosure herein.

The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed, and such modifications and variations are considered to be within the scope of this disclosure.

For conciseness, several aspects or embodiments are described herein as genera and/or as lists of alternative species. In each instance, subgenera and individual species are contemplated as individual aspects or embodiments of the invention. For aspects described as ranges, integer and half-integer subranges are specifically contemplated.

All references, including publications, patent applications, and patents, as well as database entries (e.g., sequence database entries) cited herein are hereby incorporated by reference in their entirety and to the same extent as if each reference were individually and specifically indicated to be incorporated by reference and were set forth in its entirety herein (to the maximum extent permitted by law). All headings and sub-headings are used herein for convenience only and should not be construed as being limiting in any way. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. The citation and incorporation of patent documents herein is done for convenience only and does not reflect any view of the validity, patentability, and/or enforceability of such patent documents.

1. A method for treating a subject presenting with CD47+ disease cells, comprising administering to the subject a treatment-effective combination comprising (1) a CD47-binding form of SIRPαFc, and (2) an anti-VEGF agent.

2-7. (canceled)

8. The method according to claim 1, wherein the anti-VEGF agent is an anti-VEGF antibody or an anti-VEGF receptor antibody.

9. The method according to claim 8, wherein the anti-VEGF agent is an anti-VEGF antibody.

10. The method according to claim 9, wherein the anti-VEGF agent is bevacizumab or a VEGF-binding fragment or variant of bevacizumab.

11-12. (canceled)

13. The method according to claim 8, wherein the anti-VEGF agent is an anti-VEGF receptor antibody.

14. The method according to claim 13, wherein the anti-VEGF agent is an anti-VEGFR-2 antibody.

15-16. (canceled)

17. The method according to claim 1, wherein the SIRPαFc comprises the IgV region of human SIRPα variant 2.

18. The method according to claim 17, wherein the SIRPαFc fusion protein comprises the amino acid sequence of SEQ ID NO: 3.

19. The method according to claim 17, wherein the SIRPαFc fusion protein comprising comprises the amino acid sequence of SEQ ID NO: 8.

20. The method according to any claim 1, wherein the CD47+ disease cells comprise CD47+ cancer cells.

21. The method according to claim 20, wherein the CD47+ cancer cells are blood cancer cells or solid tumour cells.

22. The method according to claim 20, wherein the CD47+ cancer cells comprise blood cancer cells.

23-28. (canceled)

29. The method according to claim 21, wherein the cancer cells comprise solid tumour cells.

30. The method according to claim 29, wherein the solid tumour cancer cells comprise breast cancer cells, lung cancer cells, or ovarian cancer cells.

31-32. (canceled)

33. The method according to claim 29, wherein the solid tumour CD47+ cancer cells are selected from colorectal, renal, hepatocellular carcinoma and glioblastoma cells.

34. (canceled)

35. A combination of anti-cancer agents, comprising an amount of a CD47 binding form of SIRPαFc, and an amount of bevacizumab effective, in combination, to deplete CD47+ cancer cells, together with instructions teaching the use thereof according to claim 1.

36. The use of the combination according to claim 35, for the treatment of a subject presenting with CD47+ disease cells.

37-40. (canceled)

41. The use according to claim 36, wherein the CD47-blocking from of SIRPαFc comprises SEQ ID NO: 3.

42. The use according to claim 36, wherein the CD47-blocking from of SIRPαFc comprises SEQ ID NO: 8.

43. comprising unit dose formulations of a CD47-binding form of SIRPαFc, and an anti-VEGF agent.

44. The kit according to claim 43, wherein the CD47-binding form of SIRPαFc, and an anti-VEGF agent are packaged together but not in admixture.
